(19) United States
(12) Patent Application Publication
(10) Pub. No.: US 2024/0366118 A1
(43) Pub. Date: Nov. 7, 2024

(54) Methods And Devices For Non-Invasive Measuring Of Blood Glucose Using Focused Light Sources

(71) Applicant: ASTELLAR LABS, lNC., Providence, RI (US)

(72) Inventors: Arvind Sai Veluvali, San Francisco, CA (US); Cutler Bozeman Whitely, Cambridge, MA (US)

(21) Appl. No.: 18/557,925

(22) PCT Filed: Apr. 29, 2022
(86) PCT No.: PCT/US2022/027095

§ 371 Date: Oct. 27, 2023

Related U.S. Application Data
(60) Provisional application No. 63/181,508, filed on Apr. 29, 2021.

(51) Int. Cl. A61B 5/145; A61B 5/00; A61B 5/1455; G16H 10/40

(52) U.S. Cl. CPC A61B 5/14532; A61B 5/0002; A61B 5/0095; A61B 5/14551; A61B 5/6838; A61B 5/7264; A61B 5/742; G16H 10/40

(57) ABSTRACT

This invention provides methods and devices for the non-invasive measurement of select substances in human tissue such as, for example, blood glucose. The non-invasive methods and devices use a light source such as a laser diode for transmitting light energy pulses into human tissue. A piezoelectric component and/or ultrasonic transducer detects vibration of the tissue and generates an acoustic signal that is transmitted to a microcontroller. The concentration of the substance, for example, blood glucose is measured using at least one algorithm. Preferably, the device incorporates embedded machine learning (ML), artificial intelligence (AI), internet of things (IoT), app, and blockchain programming, wherein the programming is designed to encrypt and/or deidentify personal data and measurements.

### CROSS-REFERENCE TO RELATED APPLICATIONS

The application claims the benefit of PCT International Application No. PCT/US2022/027095 filed Apr. 29, 2022, which claims the benefit of U.S. Provisional Patent Application No. 63/181,508 filed Apr. 29, 2021, the entire disclosures of which are hereby incorporated by reference.

### BACKGROUND OF THE INVENTION

### Field of the Invention

The present invention relates generally to methods and devices for the non-invasive measurement of blood glucose. More particularly, the invention relates methods and devices for the non-invasive measurement of blood glucose using focused light sources.

### Brief Review of the Related Art

In recent years, medical researchers have developed new methods for monitoring blood glucose concentration. These methods are particularly helpful for diabetic patients. Diabetes is a serious disease that can lead to patients having problems with many body organs and systems including, for example, kidneys, heart, nerves, blood vessels, eyes, and the like. Patients having diabetes need to monitor their blood glucose levels frequently in order to maintain a proper level of insulin in their blood. By testing blood glucose levels often, diabetics can better manage their medication, diet, and exercise.

One invasive procedure for monitoring blood glucose levels involves pricking the finger of a patient to obtain a blood sample. Then, the blood sample is analyzed and the glucose concentration is measured using an enzyme-based method. On the other hand, non-invasive blood glucose monitoring does not involve a finger-prick or other body invasive procedure for obtaining a blood sample. Rather, non-invasive techniques involve using optical technology for monitoring a blood sample inside of the body. For example, fluorescence spectroscopy, Raman spectroscopy, surface plasmon resonance, optical coherence imaging, optical polarimetry, and near-infrared spectroscopy, and the like can be used. Most conventional non-invasive methods use incident light radiation that can penetrate body tissue to monitor the blood circulating in the tissue. Correlations are made between the light absorption and blood glucose concentration.

Some commercial glucose measurement devices include FreeStyle Libre® (Abbott Diabetes Care, Inc., Alameda, CA) or the Dexcom G6 CGM® (Dexcom, Inc., San Diego, CA). Glucose measurement can also be done at-will using handheld glucometers. Both of these conventional glucose measurement regimes are invasive. Some conventional glucose measurement device rely on some portion of the device, for example, needle, microchip, monitor, and the like residing continuously within the patient's body. Other glucose measurement systems involve the use of finger sticks intended to draw small quantities of blood from the patient.

More particularly, in photoacoustic (PA) systems, a light source is used to generate a pulsed concentrated light beam which is subsequently absorbed by human tissue. Upon contact with the human tissue, the tissue generates heat and thermally expands. In responses to this thermal expansion, ultrasonic waves are generated by the excited tissue. These ultrasonic waves are then collected by a high frequency (>1 MHz) ultrasonic transducers or piezoelectric components that can display the data visually by a range of amplitude

Although these photoacoustic systems are generally effective, some of these systems have some drawbacks. For example, one problem with many conventional techniques is they do not compensate for the absorption of the light radiation by water. Body tissue normally has a high level of water, and this can prevent a light beam from sufficiently penetrating the tissue to be absorbed by the blood. Also, the water can have a higher absorption of the light beam radiation over the blood glucose, and this can cause an acoustic signal that interferes with the signal from the blood glucose.

Another drawback with some conventional photoacoustic systems is they can include some complex components that increases manufacturing time and costs. There is a need for new methods and devices for the non-invasive measurement of blood glucose. These methods and devices should use specific light beam irradiation spectra for absorption by glucose and that also accounts for water absorption. These methods and devices also should be cost-effective for manufacturers and consumers. There is also a need for a more efficient systems that can connect easily communicate with mobile and/or web application. The present invention that provides such non-invasive systems. For example, the systems of the present invention use embedded machine learning [ML] and artificial intelligence [AI] have (Internet of Things [IoT] capabilities) for that can serve as a portal into back-end mobile and web applications and provide hand-held preventive and personalized medicine. The present invention utilizes both AI and IoT embedded printed circuit boards (PCBs), Fabrey-Perot (FP) laser diodes, lithium polymer (Li—Po) batteries, and compact ground (OLED) screens to make the devices economically feasible for all parties. Other features, advantages and benefits of the present invention are described further below.

### SUMMARY OF THE INVENTION

The present invention relates generally to a device capable of detecting and measuring select substances, for example, glucose, in human tissue. The device measures the substances without extracting a sample of said tissue from a user of the device. In one preferred embodiment, the device utilizes optical technology including directing incident light radiation to penetrate the body tissue of the user. In turn, the irradiated body tissue generates an acoustic wave. The device should compensate for the absorption of light radiation emitted by water and substances contained in the user.

In one preferred embodiment, the device measures glucose levels in human blood a user. Particularly, the user can be a patient suffering from a disease caused by abnormal insulin levels in the patient's blood. More particularly, the patient can be suffering from a disease, for example, selected from the group consisting of Type I or Type II diabetes, obesity, insulin resistance, high blood pressure, peripheral neuropathy, cardiovascular disease, metabolic syndrome, kidney disease, nephropathy, stroke, Alzheimer's disease, hepatopathy, and combinations thereof. The device preferably comprises machine learning programming and/or artificial intelligence programming. Particularly, the device preferably incorporates embedded machine learning (ML), artificial intelligence (AI), internet of things (IoT), app, and blockchain programming, wherein said programming is designed to encrypt and/or deidentify personal data and measurements.

In one particularly preferred embodiment, the device comprises one or more embedded circuit boards; one or more sources of light; a microcontroller or microprocessor comprising an algorithm; an ultrasonic transducer, wherein said ultrasonic transducer is designed to measure acoustic energy; a power source; and a means for displaying the substance measurement value. The device can further comprise a chassis. In one example, the chassis comprises an upper clamping arm and a lower clamping arm connected by a spring biasing means, and the chassis secures the device to the patient. The clamping arms can further comprise grooves so that the user can insert and rest his/her finger or other body part in the clamping arms. Different clamping means can be used to close the clamping arms and apply the force needed to secure the finger or other body part in the device. For example, the clamping means can be selected from the group consisting of a coil, spring, helical compression spring, extension spring, flat spring, torsion spring, clamp, hinge, pin, adhesive, gel, elastomeric material, and combinations thereof.

Different light sources can be used. For example, a light source selected from the group consisting of a laser, laser diode, light-emitting diode, photodiode, an electrogenerated chemiluminescence-200 (ECL-200), multichannel light source-8000 (MLS-8000) and combinations thereof can be used. In one preferred embodiment, the laser diode is a Fabrey-Perot laser diode that transmits light as pulses. The pulses can be emitted at a frequency between 1 to 10 Hz±5%. The diode can be connected to a digital output pin. The device can also include a lens. Different power sources can be used. For example, the power source can be one or more batteries such as, for example, lithium polymer batteries and rechargeable batteries.

Also, different display means can be used. For example, a display screen such as, for example, an organic light-emitting diode screen can be used. The display means can be used to show a blood glucose concentration level. In one embodiment, the transducer is a piezo electric component. The transducer can be connected to a digital output pin. In one embodiment, the microcontroller further comprises a transmitter that is preferably wireless. The microcontroller also can further comprise an amplifier and/or transistor such as a bipolar junction transistor (BJT) or metal-oxide-semiconductor field-effect transistor (MOSFET). In one preferred version of the device, the microcontroller takes a baseline reading of the user's tissue. The circuit board can further comprise one or more embedded sensors selected from the group consisting of acceleration, inertial, humidity, temperature, barometric, pressure, proximity, light color and luminosity sensors, and combinations thereof. The embedded circuit board can further comprise a microphone.

The device can further comprise a wireless interface, wherein the measured substance concentration levels can be viewed on a wireless hardware piece preferably selected from the group consisting of a mobile phone, tablet, watch, wearable device, monitor, and computer. The device can further comprise a wiring interface, wherein the device is connected by the wiring interface to a vital sign monitor capable of displaying the measured substance concentration level of the user.

The device can also include other systems. For example, there can be a means for monitoring blood oxygen concentration, wherein the measured blood oxygen concentration is displayed on the vital signs monitor; a means for monitoring body temperature, wherein the measured body temperature is displayed on the vital signs monitor; and/or a means for monitoring pulse rate, wherein the measured pulse rate is displayed on the vital signs monitor.

The above-described piezoelectric component preferably has a frequency in the range of 3 to 5 MHz±5%. In one embodiment, the laser diode emits light energy having a wavelength in the range of 1550 to 1750 nm±5% and preferably the laser diode emits light energy having a wavelength of about 1600 nm±5%. The resistor preferably has a resistance of 30Ω to 35Ω±5%. The resistor can be installed in place. The piezoelectric component, laser diode light source, microcontroller, and display screen are preferably contained in a chassis assembly, the chassis assembly being adapted for holding a body part of a user.

The present invention also provides a non-invasive blood glucose measuring device, comprising an ultrasonic transducer for measuring the baseline glucose concentration of a patient, a laser diode light source for transmitting light energy pulses into a body tissue of the patient, and a microcontroller wherein the ultrasonic transducer detects vibration of the body tissue and generates an acoustic signal that is transmitted to a microcontroller, and wherein the concentration of the blood glucose is measured using at least one algorithm that determines the difference between the baseline and final blood glucose concentration levels. The intensity of the ultrasonic waves of the ultrasonic transducer is preferably related to the intensity of the continuously changing intensity of the light applied to the body tissue.

The present invention also encompasses a method for non-invasive measuring of blood glucose concentration levels in a patient, comprising the steps of: a) measuring the baseline of the resting tissue of a user; b) transmitting light energy pulses into a body tissue of the user; c) detecting the vibration of body tissue resulting from the light energy pulses being transmitted, wherein the vibrations generate an acoustic signal; and d) analyzing the acoustic signal using at least one algorithm to determine the difference between the baseline and final level of vibration, wherein the algorithm interprets the level of vibrations into the amount of blood glucose.

The method can further include the step of using a microcontroller to determine if the baseline tissue values are within a predetermined range and the algorithm proceeds if the baseline values are within this range. A laser diode can be used to transmit the light energy pulses. At least one algorithm can be used to determine a time interval for transmitting the light energy pulses and a time interval for pausing the light energy pulses. In the algorithm, the baseline (NL) data and light transmitted laser diode induced (L) data can be saved as variables that can be inserted into the algorithm equation: Signal Value=([L1+L2+L3/3)−([NL+NL2+NL3]/3).

The device is preferably attached to the body tissue of a user, wherein said body tissue is relatively thin with an available blood supply. For example, the device can be attached to a body part of the patient selected from the group consisting of interdigital folds, thenar webspace, one or more fingers, wrist, arm, ear, nostril, head, lip, tongue, neck, back, stomach, chest, genitals, and foot. Also, the arms of the chassis can be manufactured from a polymer resin injection molding process or 3-D printing process. In some embodiments, the parts selected from the group consisting of circuit boards, piezoelectric component, and laser diode, are custom made.

### BRIEF DESCRIPTION OF THE DRAWINGS

The novel features that are characteristic of the present invention are set forth in the appended claims. However, the preferred embodiments of the invention, together with further objects and attendant advantages, are best understood by reference to the following detailed description in connection with the accompanying drawings in which:

FIG. 1 is a block flow diagram of one embodiment of the present invention showing a non-invasive method for detecting the concentration of glucose in the blood;

FIG. 2 is a perspective view of one embodiment of a chassis of the present invention;

FIG. 2A is a side view of one embodiment of a chassis of the present invention;

FIG. 3 is a schematic side view diagram showing a patient's finger inserted into the chassis;

FIG. 4 is a schematic top view diagram showing a patient's finger inserted into the chassis;

FIG. 5 is a perspective view of the one embodiment of a chassis of the present invention showing the clamping arms;

FIG. 5A is a top view of the one embodiment of a chassis of the present invention showing a first clamping arm;

FIG. 5B is a top view of the one embodiment of a chassis of the present invention showing a second clamping arm;

FIG. 5C is a schematic top view diagram showing one embodiment of a chassis of the present invention secured to the webspace/thenar space of a patient's hand;

FIG. 6 is a schematic diagram showing the components and circuitry of a first embodiment of the chassis of the present invention;

FIG. 7 is a first exploded view of one embodiment of the present invention showing the components and circuitry of the chassis;

FIG. 8 is a second exploded view of one embodiment of the present invention showing the components and circuitry of the chassis;

FIG. 9 is a block flow diagram showing one embodiment for an algorithm that can be used for detecting the concentration of glucose in the blood in accordance with the present invention.

FIG. 10 is a schematic diagram showing the components and circuitry of a second embodiment of the chassis of the present invention;

FIG. 11 is an exploded view of one embodiment of the present invention showing the components and circuitry of the chassis;

FIG. 12 is a first perspective view of one embodiment of the chassis of the present invention; and

FIG. 13 is a second perspective view of one embodiment of the chassis of the present invention.

### DETAILED DESCRIPTION OF THE INVENTION

The device and methods according to the present invention involve the light-radiation of body tissue and this interaction of the light energy with the tissue causes an acoustic signal to be generated. This interaction is used to determine the concentration of glucose within the blood tissue.

When a pulsed beam of light radiation passes through the human body tissue, energy is absorbed and there is heating of the tissue. As the light beam is absorbed, this causes periodic heating of the body tissue. The radiation is focused on the surface of the tissue. This results in the tissue expanding and vibrating. This expansion causes an acoustic wave to be generated. The size of the generated acoustic wave is directly related to the amount of energy absorbed in the tissue medium from the light beam. Thus, the measured acoustic signal of the tissue is a function of the wavelength of the light beam that penetrates and is absorbed by the tissue.

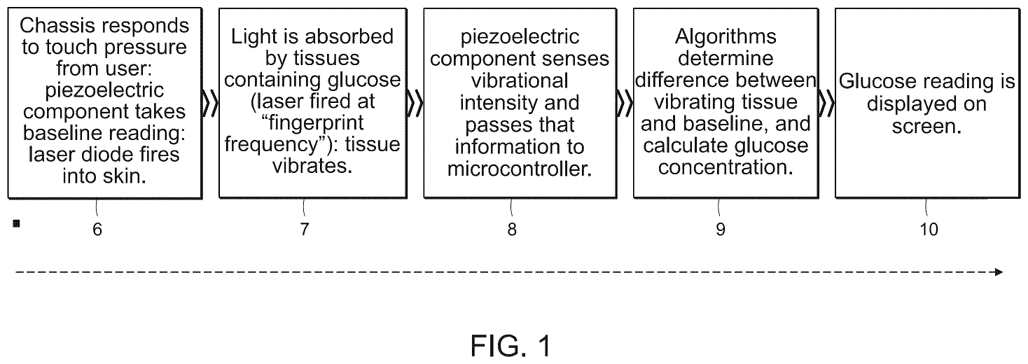

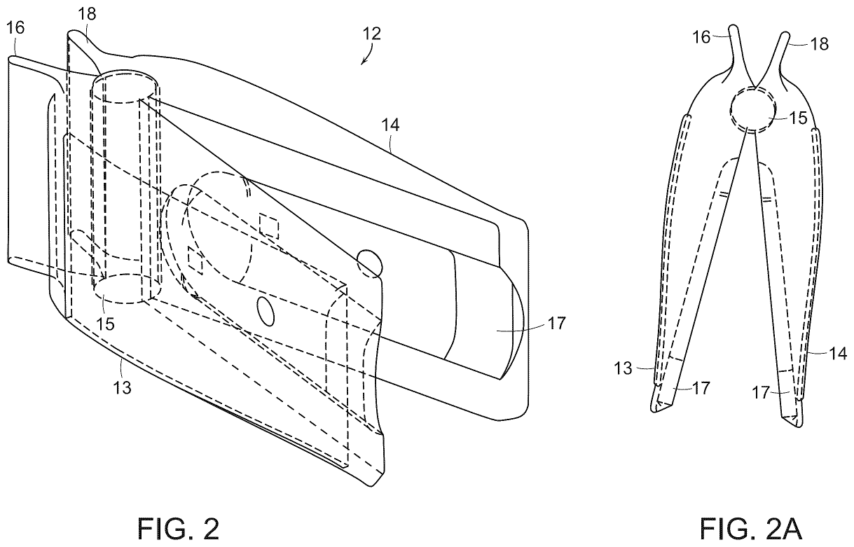

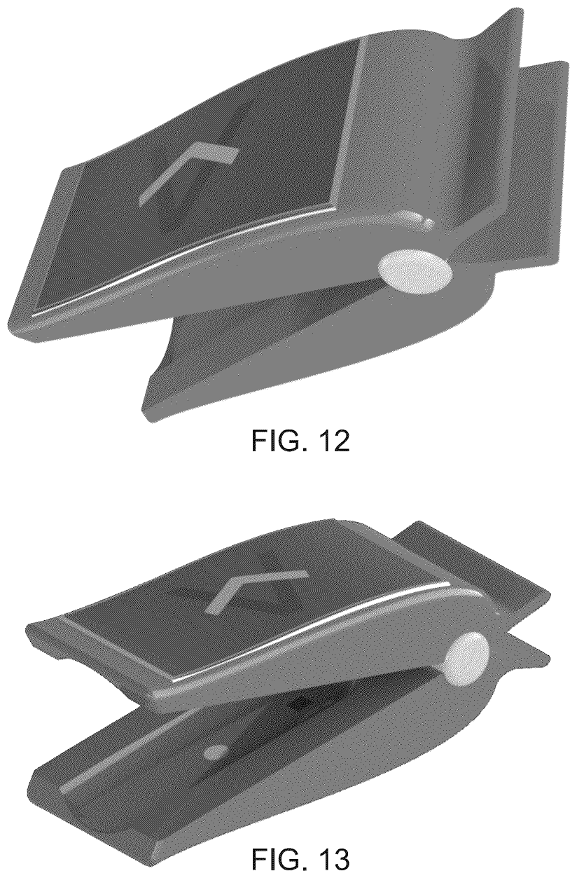

Referring to FIG. 1, a block flow diagram of one embodiment of a non-invasive method for detecting the concentration of glucose in the blood in accordance with the present invention is shown and generally indicated at (4) with the specific steps indicated at (6-10). As shown in FIGS. 2 and 2A, in one embodiment, the system includes a chassis (12) having upper and lower clamping arms (13, 14) and these arms are forced together by a spring biasing means (15). In one embodiment, the spring (15) is a coil spring. Different clamping means can be used to close the clamping arms and apply the force needed to secure the finger or other body part in the device. For example, the clamping means can be selected from the group consisting of a coil, spring, helical compression spring, extension spring, flat spring, torsion spring, clamp, hinge, pin, adhesive, gel, elastomeric material, and combinations thereof. Referring to FIGS. 12 and 13, other perspective views of the chassis showing the clamping arms are shown. The chassis can be manufactured from a polymer resin injection molding process or 3-D printing process.

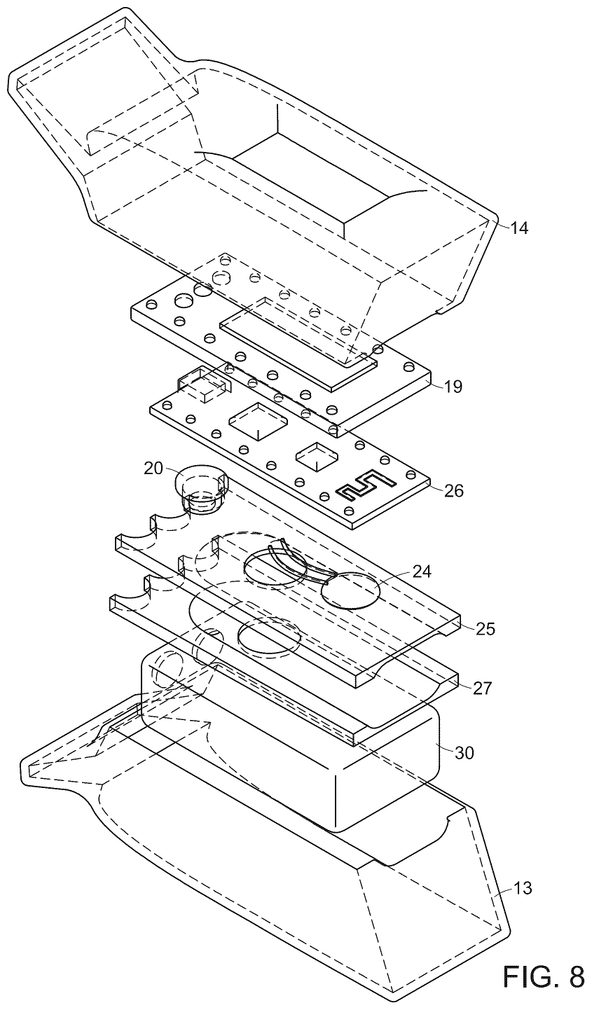

The clamping arms (13, 14) contain grooves (17) to provide a snug and comfortable fit, to protect the user, and to reduce ambient noise from the outside environment. A screen (19) (FIGS. 7 and 8) is situated on the upper clamping arm of the device. The screen displays a welcoming message and a measured reading immediately after use as described further below. It is recognized that in other embodiments of this invention, a chassis does not need to be used.

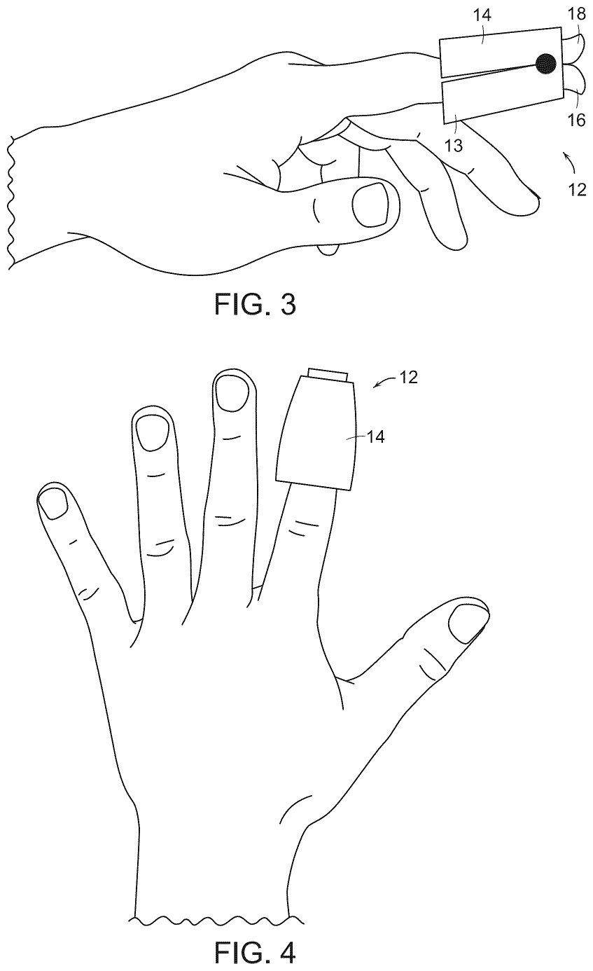

Turning to FIGS. 3 and 4, a patient's finger is inserted between the upper and lower clamping members (13, 14) and placed in the grooved channels (17). The clamping members apply a clamping force so that the chassis (12) is firmly secured to the finger. When the patient or health care provider pinches the upper and lower handle members (16, 18) so the clamping force is no longer applied, then the finger can be removed easily from the chassis (12).

### Hardware and Circuitry of Chassis

In one preferred embodiment, the Printed Circuit Board (PCB) used in the chassis device (12) is the Arduino® Nano 33 BLE Sense (Arduino® SA societé anonyme, Lugano, CH). This PCB is Arduino®'s 3.3 V (Input Vₘₐₓ is 21 V) Artificial Intelligence (AI) enabled board having dimensions that are only 45×18 mm. Preferably, the device of the present incorporates embedded machine learning (ML), artificial intelligence (AI), internet of things (IoT), app, and blockchain programming, wherein said programming is designed to encrypt and/or deidentify personal data and measurements. Preferably, the components embedded on the PCB include 9 axis inertial sensors, humidity and temperature sensors, a barometric sensor, a microphone, as well as a pressure, proximity, light color and light intensity (i.e., luminosity) sensors. This array of sensors makes the board ideal for wearable devices by sensing motion, reducing data noise by accommodating environmental conditions, capturing voice commands, estimating ambient luminosity, and sensing when the user is in proximity to the device.

This board also contains the nRF52840 processor (Nordic Semiconductors®, Trondheim, Norway), a 32-bit ARM Cortex-M4 CPU running at 64 MHz which is used primarily for handling larger programs with many more variables. This processor also includes Bluetooth® Low Energy (BLE) (Bluetooth Sig, Inc., Washington, DC) pairing via Near-Field Communication (NFC) and ultra-low power consumption modes used for communicating securely with other devices. Edge computing applications for Artificial Intelligence (AI) are also made possible on this device using TinyML® (TinyML Foundation, Los Altos, CA) and Machine Learning (ML) models can be created using TensorFlow® Lite (Google, Inc., Mountain View, CA) by uploading them via the Arduino® IDE (Arduino® SA societé anonyme, Lugano, CH). Code is uploaded to the microcontroller using a microUSB connection which also serves as a power source; however, Lithium Polymer (Li—Po) batteries (e.g., Adafruit® 3.7 V, 400 mAh Li—Po battery, (Adafruit Industries, New York, NY)) can be directly connected to the voltage in (VIN) and ground (GND) pinouts on the device to serve as a portable, rechargeable energy source for the handheld device. Lastly, the communications chipset on the device can be both a Bluetooth and BLE client and host device operating as either a central or peripheral device.

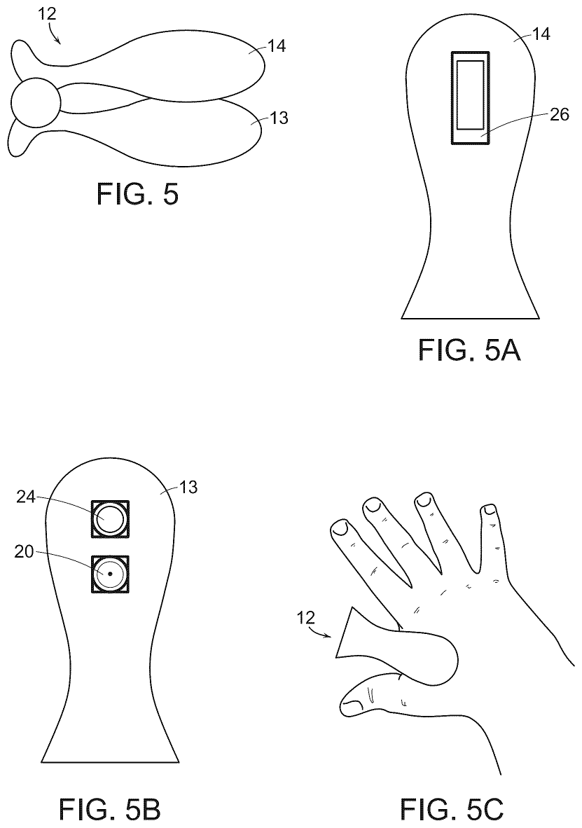

Referring to FIGS. 5 to 5C, different components and circuitry of the chassis (12), particularly the clamping arms (13, 14) are illustrated. The components include a laser diode (20), a transducer (24), and a microcontroller (26). In some embodiments, the parts selected from the group consisting of circuit boards, piezoelectric component, and laser diode, can be custom made. Although the chassis (12) is normally placed on a patient's finger, it is recognized the chassis can be applied to other body parts such as, for example, an ear or the webspace/thenar space of the hand (FIG. 5C). Preferably, the body tissue is relatively thin and has a good blood supply because it is easier to make take accurate measurements on such tissue.

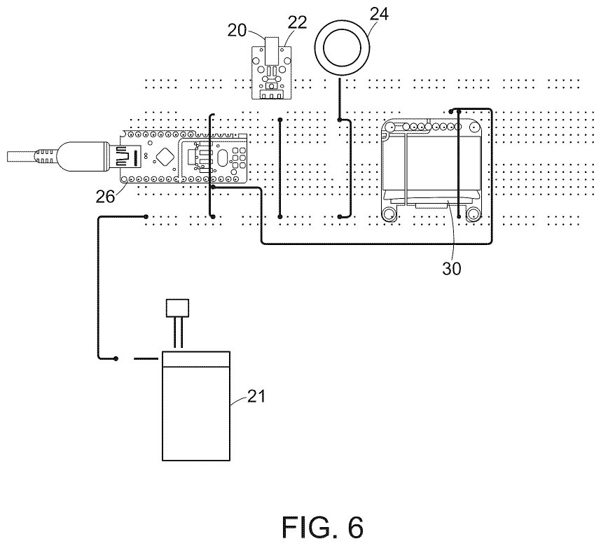

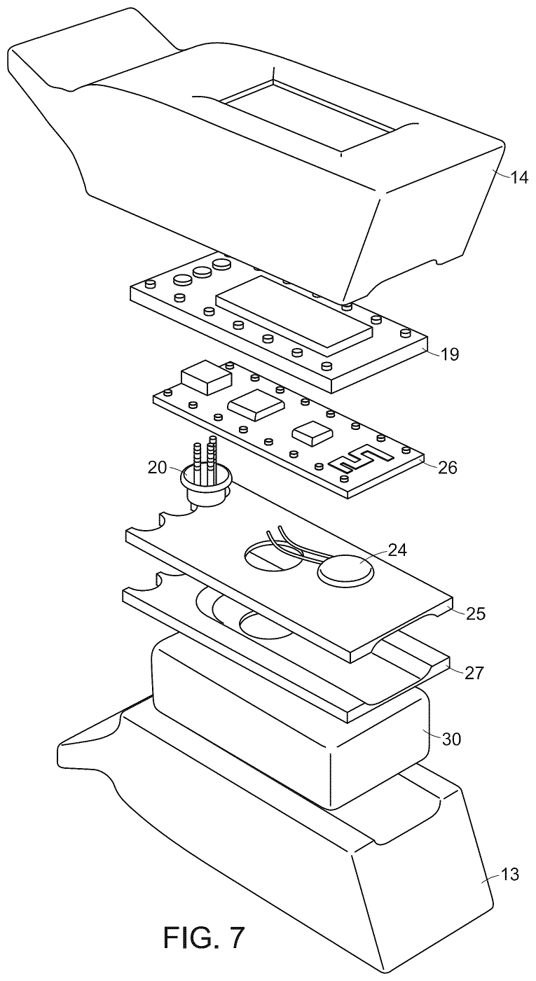

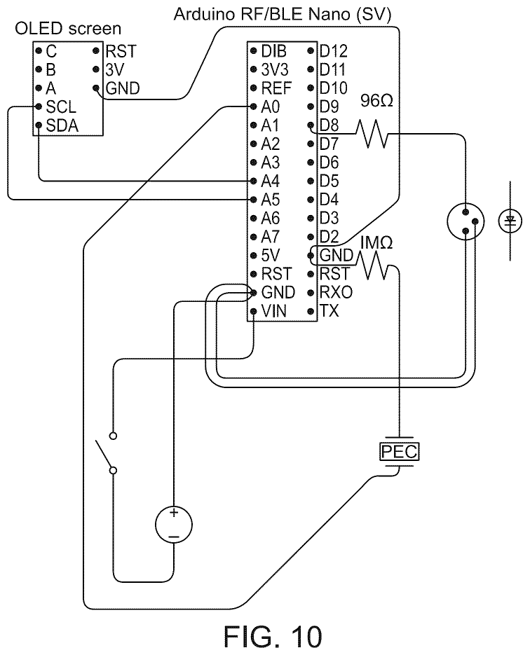

In FIGS. 6 and 10, the different components and circuitry of different embodiments of the chassis (12) are illustrated in further detail. A laser diode (20) is preferably as the light source and is controlled to emit the light in pulses at a frequency generally in the range of about 1 to about 10 kHz. Suitable examples of laser diodes that can be used in accordance with this invention are described further below. The laser diode (20) is driven by a power supply (21). Different power sources can be used. For example, the power source can be one or more batteries such as, for example, lithium polymer batteries and rechargeable batteries. The laser diode (20) transmits light energy directly onto the skin. A lens (22) can be used to concentrate the incident light beam. Once the skin is irradiated and the light energy is absorbed, acoustic energy is generated. This acoustic signal is used to determine the concentration of glucose or other substance within the blood flowing through the body tissue. More particularly, as the light beam from the laser diode or other light source is absorbed by the body tissue, the tissue expands and vibrates. This expansion and vibration of the body tissue generates an acoustic wave. The size of the acoustic signal is related to the amount of light energy absorbed by the body tissue. Once this absorption level has been determined, the blood glucose concentration or other concentration of substances can be determined using algorithms as described further below.

A transducer (24) can be used to detect the acoustic energy resulting from the light absorption and it then generates an electric signal. An amplifier can be used to amplify the signal. Piezoelectric transducers may be used. Suitable examples of transducers that can be used in accordance with this invention are described in further detail below. At each pulse of light energy and as the energy is absorbed, an acoustic signal is generated in the body tissue. The signal from the transducer can pass through an amplifier and can be measured and stored in the microcontroller (26).

Turning back to FIG. 1, in this first step (6) of one embodiment of the method of this invention, the piezoelectric transducer (24) takes a baseline reading of the body tissue and blood glucose concentration; and the light source (20) is switched on to emit light energy and irradiate the targeted body tissue. This first step (6) is described in further detail below.

### Laser Diode

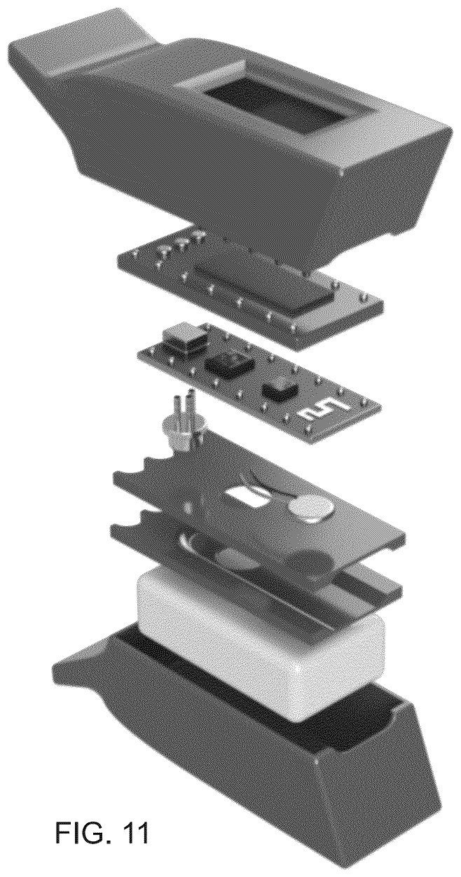

Turning to FIGS. 7 and 8, exploded views of one embodiment of the present invention showing the components and circuitry of the chassis (12) are shown. Referring to FIG. 11, another exploded view of the components and circuitry is shown. one embodiment of the different components and circuitry of a second embodiment of the chassis (12) are illustrated in further detail. In one preferred embodiment, a 1600 nm Fabry-Perot (FP) laser diode (LD) model FB (Laser Diode Source [Fibercom™, Ltd], Bozeman, MT) is used in the chassis of this invention. The laser diode (LD) is indicated at (20). The LD source is connected to a digital output pin (D2-12) (Arduino® SA societé anonyme, Lugano, CH) and ground, whereby its pulsatile functionality can be controlled by programming uploaded to the microcontroller via the Arduino® IDE (Arduino® SA societé anonyme, Lugano, CH). The microcontroller is indicated at (26). The digital output applies discrete ON and OFF functionality to promote this pulsatility. Based on the LD specifications, a resistor serves to reduce current flow, adjust signal levels, divide voltage, bias active elements, and terminate transmission lines is used. The resistor is dependent upon voltage and current being received relative to the LD baseline operating specifications. In one preferred embodiment, a 30 to 35 (preferably, 32.14) Ω resistor (Vishay Dale™ Electronics, Inc., Columbus, OH) is used.

Piezoelectronic Component and/or Ultrasound Transducer

The piezoelectric component (PEC) and/or ultrasound transceiver (UST) is wired to an analog (A0-7) and ground (GND) pinout located on the Arduino® Nano 33 BLE sense PCB (Arduino® SA societé anonyme, Lugano, CH). The piezoelectric component (PEC) and/or ultrasound transceiver (UST) are indicated at (20) and (24). Wiring the PEC and/or UST to an analog sensor allows the transceiver to receive data in a range of applied voltages continuously over time. The intensity of ultrasonic waves of the UST is directly proportional to the intensity of the pulsatile or continuously changing intensity of the light applied to the tissue.

### Screen

In one preferred embodiment, a Adafruit® 128×64 ground organic light-emitting diode (OLED) FeatherWing® display screen (Adafruit® Industries, New York, NY) is used in the device (chassis) of this invention. The screen is indicated at (19). The screen is made of 128×64 individual white organic light-emitting diode OLED pixels. The display also makes its own light, so no additional backlight is required, reducing the power needed to run the OLED. This also contributes to the screen's crispness and high contrast. This screen uses only an inter-integrated circuit (I2C) so it can be connected with just two pins (plus power and ground) and there is also an auto-reset circuit and reset button located at the top of the screen. This display will draw 10 mA when in use and will use 3V power and logic. For this device, the screen is simply connected to the 3.3 V and ground (GND) pins from Arduino® (Arduino® SA societé anonyme, Lugano, CH). Data transfer occurs over the I2C pins SDA (data line) and SCL (clock line)—there are two 2.2K pull ups to 3 V on each. Three buttons are situated on the left side of the screen to be programmed for the user interface. Finally, there is a restart button available which allows the user to reset the entire device.

### Other Light Sources

As discussed above, in one preferred embodiment, a 1600 nm Fabry-Perot (FP) laser diode (LD) model FB (Adafruit® Industries, New York, NY) is used as the light source. Other suitable light sources include lasers, light emitting diodes (LED), and combinations thereof. Multiple lasers also can be used. In one example, an electrogenerated chemiluminescence-200 (ECL-200), which is a manual tunable LD light source unit developed for multi-light sources on the basis of technologies accumulated by Santec™ Corporation (Komaki Aichi, JP) external cavity type semiconductor laser light source series, can be used. The ECL-200 is a wavelength stable light source wherein a light grid filter arranged inside the light source serves as the standard wavelength type. High-precision temperature control of the light grid filter to produce this standard wavelength has resulted in highly stable wavelength production and repeatability. Up to eight channels of ECL-200 can be loaded onto the multi-channel light source (MLS) power source rack, the MLS-8000, which enables the user to use the ECL-200 as a light source to evaluate wavelength-division multiplexing (WDM) communication and so forth. Given that this device produces a continuous wave laser, the pulsatile laser can be produced using an optical chopper.

In another example, the multichannel light source-8000 (MLS-8000) (Santec™ Corporation, Komaki Aichi, JP), which is a power source rack for multichannel light sources, can be used. Up to eight channel light source units may be loaded on the MLS-8000, and it enables users to be able to configure their optimized multi-channel light source by combining various light source units into one external cavity. The MLS-8000 is also equipped with a general purpose-interface Bus (GP-IB) output for two-way communication between the device and the user, making it simple for users to completely control specific light source units externally.

As discussed above, there will likely be some unavoidable background noise generated by both known and unknown origins being collected by the ultrasound transceiver (UST) (likely due to biomolecules unaccounted for or ambient pressure from the environment/user), ML and possibly AI will be required to differentiate between true signals from false positives. Thus, in one preferred embodiment, the printed circuit boards (PCB) contain embedded microcontrollers capable of ML and AI directly within the device. In one embodiment, the Arduino® Nano 33 BLE Sense PCB (Arduino® SA societé anonyme, Lugano, CH) is used. Other PCBs that can be used in accordance with this invention include, but are not limited to, the Arduino® Vidor MKR 4000 (with field programmable gate arrays (FPGA)), the Adafruit® Feather nRF52840 Sense® (Adafruit Industries, New York, NY), and the Arduino® Nano 33 BLE Sense (Arduino® SA societé anonyme, Lugano, CH). The Arduino® Nano 33 BLE Sense is particularly effective, because it has good cost effectiveness, size, capabilities, and robust documentation. The sensors are already embedded in the PCB itself (i.e., temperature, accelerometer, and barometric pressure sensors) as well as Bluetooth® Low Energy (BLE) which allows it to connect with outside devices securely and wirelessly (e.g., a mobile phone). Additionally, the PCB contains an nRF52840 microcontroller which is capable of harnessing and embedding the ML and AI powers of TensorFlow® Lite (Google, Inc., Mountain View, CA) onto the hand-held device-itself also securely encrypted. Finally, robust documentation and an extensive integrated development environment (IDE) was available which uses the C++ (primarily) and Python® languages (partnership with TensorFlow® Lite (Google, Inc., Mountain View, CA)), making the backend programming much simpler and more capable by providing the option of coding with a language appropriate each respective application. The IDE also displays real time data through a serial plotter, making instant data analysis possible. Preferably, the device of the present incorporates embedded machine learning (ML), artificial intelligence (AI), internet of things (IoT), app, and blockchain programming, wherein said programming is designed to encrypt and/or deidentify personal data and measurements.

The importance of the ancillary sensors embedded onto the PCB, as stated before, serve to possibly enhance the versatility for future wearable use (e.g., accelerometer for “waking” the device up prior to using it), and to account for extraneous background noise (e.g., temperature and pressure sensors) without having to add additional hardware that will give unwanted bulk to the product and increase expense; however, if at any point these sensors do not prove to be useful, our own customized PCB will be designed space-save and cut out unnecessary cost.

Furthermore, Bluetooth® Low Energy (BLE) or some form of wireless communication capabilities (e.g., WiFi) will be required in order for this device to serve as just one component of a larger network of components that can pass data along to an IoT cloud service and database (e.g., Amazon® Web Services IoTR) and integrate this data with a mobile and web application client.

As shown in the block diagram of FIG. 1, in the second step of the method (7), each consecutive pulse, the wavelength of the light incident on the patient's tissue is absorbed and the tissue vibrates. More particularly, the specific molecular composition and structure of the glucose means that it will absorb light energy at specific frequencies. This pattern of light absorption can help identify the glucose molecules. When the light is absorbed by the tissue, these molecules are heated. The vibrating molecules generate an acoustic wave. From this acoustic signal, it can be determined how much light energy, at the specific frequency, that was absorbed. The size of the generated acoustic wave is directly related to the amount of light energy absorbed in the tissue medium from the light beam. In this manner, the concentration of glucose can be measured. That is, the strength of the absorption depends upon the concentration of the glucose at the time when the light energy is absorbed.

The absorbency fingerprint of glucose lies within the near infrared (NIR) range (in the ranges of about 1450 to about 1850 nm and about 2050 to about 2400 nm). The absorbency and subsequent photoacoustic effect on the body tissue will differ and this will produce a signal that is reflective of glucose concentrations. In particular, one of the glucose absorption spectrums falls specifically between the 1550-1750 nm wavelengths. Although there are other wavelength spectrums that could have been considered, it has been found in accordance with this invention, that this spectrum-particularly at the 1600 nm wavelength-lies outside of the absorption spectrums for other biomolecules, including water. As discussed above, one problem with water and other components such as, for example, muscle, bone, fat, and fluids) in the tissue is that these materials can be sensed as opposed to the blood. Such other components can harmfully influence and alter the glucose measurement. The acoustic signals that are generated from these components can be much greater than the signals generated from the blood glucose. These other components and high background noise can cause misleading data and poor blood glucose measurement to be recorded.

As discussed above, at each radiation pulse, the molecules of the components in the tissue absorb the incident light energy. The molecules that absorb the light at this frequency are heated and expand; and this vibration generates an acoustic signal. The transducer (24) transmits that acoustic signal through the amplifier and to the microcontroller (26). (Step 8 of the block diagram of FIG. 1.)

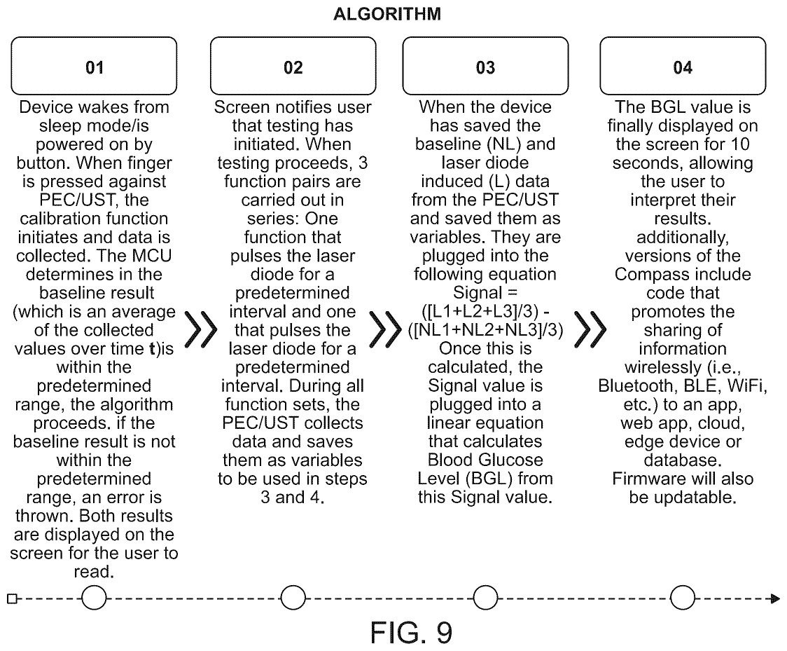

As described in Steps 9 and 10 of the block diagram of FIG. 1, once this information is passed to the microcontroller (26); then an algorithm can be used to determine the difference between the glucose concentration of the vibrating tissue and baseline glucose concentration. In this manner, the blood glucose concentration is determined. The resulting information is displayed on the screen (30). These algorithms are discussed further below and illustrated in FIG. 9.

In one embodiment, the device includes the following components: a) Piezoelectric undersaddle transducer (UST) (3-5 MHz); b) a 1600 nm laser diode (puse tau>5 ns/operating voltage of about 10 mW; operating current of less than 80 mA (preferably about 63 mA; a microcontroller (operating voltage of 3.3 V/DC current per I/O pin of 15 mA). To account for the low current input by the microcontroller (providing 15 mA) to operate the laser diode (requiring 63 mA), a transistor (e.g., bipolar junction transistor (BJT) or metal-oxide-semiconductor field-effect transistor (MOSFET)) is used. This increases the current being supplied by the microcontroller. Furthermore, a resistor (which is placed in series) will be added to the laser diode to provide the proper voltage to the laser diode. In one preferred embodiment, a 30 to 35 (preferably, 32.14) Ω resistor is used.

Referring to FIG. 7, in one embodiment of the algorithm, during the First Step the device wakes from sleep mode or is powered on by a button. When a finger is pressed against the Piezoelectronic component (PEC)/Ultrasonic Transducer (UST), the calibration function initiates and data is collected. The Microcontroller Unit (MCU) determines if the baseline result (which is an average of the collected values over time (t)) is within the predetermined range. If the baseline result is within the predetermined range, the algorithm proceeds. If the baseline result is not within the predetermined range, an error is thrown. Both results are displayed on the screen for the user to read.

In Step 2 of FIG. 7, the screen notifies the user that testing has been initiated. When testing proceeds, three (3) function pairs are carried out in series. One function is used that pulses the laser diode for a predetermined interval and one function that pauses the laser diode for a predetermined interval is used. During all function sets, the PEC/UST collects data and saves them as variables to be used in Steps 3 and 4.

In Step 3 of FIG. 7, when the device has saved the baseline (NL) and laser diode induced (L) data from the PEC/UST and saved them as variables. They are plugged into the following equation Signal=([L1+L2+L3/3)−([NL+NL2+NL3]/3). Once this is calculated, the Signal Value is plugged into a linear equation that calculates Blood Glucose Level (BGL) from these Signal Values.

In Step 4 of FIG. 7, the BGL value is finally displayed on the screen for ten (10) seconds, allowing the user to interpret their results. Additionally, versions of the present invention include code that promotes the sharing of information wirelessly (i.e., Blutooth®, BLE, WIFI, and the like) to an App, Web App, Cloud, Edge Device, or Database. Firmware will also be updatable.

The present invention is further illustrated by the following Example, but this Example should not be construed as limiting the scope of the invention. For instance, the device of the present invention is not limited to measuring blood glucose levels, but it may also be used to measure the concentration of other substances and biomolecules in human body tissue. In other instances, the device can also include other systems. For example, there can be a means for monitoring blood oxygen concentration, wherein the measured blood oxygen concentration is displayed on the vital signs monitor; a means for monitoring body temperature, wherein the measured body temperature is displayed on the vital signs monitor; and/or a means for monitoring pulse rate, wherein the measured pulse rate is displayed on the vital signs monitor. Also, as described above, device can further comprise a wireless interface, wherein the measured substance concentration levels can be viewed on a wireless hardware piece preferably selected from the group consisting of a mobile phone, tablet, watch, wearable device, monitor, and computer. The device can further comprise a wiring interface, wherein the device is connected by the wiring interface to a vital sign monitor capable of displaying the measured substance concentration level of the

### EXAMPLE

The above-described device and algorithms, as illustrated in FIGS. 1-13, were used in the following Example. The test in this Example evaluated the accuracy of the blood glucose monitoring device of the present invention compared to the commercially-available Contour™ EZ glucose monitoring system (Bayer). In the Contour™ EZ system, a lancet is used for pricking the finger of a patient to obtain a blood sample. Following this finger-pricking step, the blood sample is placed on a test strip. Then, the blood sample is inserted into the Contour™ EZ monitor and the blood glucose level is measured.

As described above, in the device of the present invention, a patient places their finger in the chassis and the baseline tissue dynamics are measured. This data includes the blood glucose concentration and this data is recorded. After ingestion of a glucose-rich meal, the patient places their finger in the chassis and the blood glucose levels (BGL) is measured and recorded. There data is reported below in the follow Tables.

### Example 1 (Table 1)—Comparison of Measurements of Blood Glucose Levels (BGL)

| | Measurements Using Contour | |
|---|---|---|
| Measurements Using Device | EZ ™ Test Strips and | |
| of Present Invention | Monitor (mg/dL) | Baseline |
| −79 | 79.33 | −63 |
| −89 | 89.67 | −67 |
| −93 | 91.33 | −61 |
| −154 | 93 | −55 |
| 0 | 0 | 0 |

As shown in the above Table 1, the blood glucose measuring device of the present invention provides similar measurements of blood glucose levels (BGL) to the commercial Contour EZ™ Test Strips and Monitor.

The methods and devices for the non-invasive measurement of blood glucose of the present invention provide several advantages over conventional systems. For example, when the system of the present invention is tested in vivo, it was shown to be >95% accurate with and without being embedded in a chassis. The components are relatively low cost and can be manufactured efficiently. The technology includes embedded Machine Learning (ML), Artificial Intelligence (AI), Internet of Things (IoT), app, and blockchain for encrypting/deidentifying personal data and measurements. It also should be understood that the present invention is not limited to measuring blood glucose levels, but it may also be used to measure the concentration of other substances and biomolecules in human body tissue.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

It is further understood that the methods, materials, constructions and devices described and illustrated herein represent only some embodiments of the invention. It is appreciated by those skilled in the art that various changes and additions can be made to the methods, materials, constructions and devices without departing from the spirit and scope of this invention. It is intended that all such embodiments be covered by the appended claims,

We claim:

1. A device capable of detecting and measuring select substances in human tissue, wherein said device measures said substances without extracting a sample of said tissue from a user of said device.

2. The device according to claim 1, wherein said device utilizes optical technology.

3. The device according to claim 2, wherein said optical technology uses incident light radiation to penetrate the body tissue of said user.

4. The device according to claim 3, wherein said irradiated body tissue generates an acoustic wave.

5. The device according to claim 1, wherein said device compensates for the absorption of light radiation emitted by water and substances contained in the user.

6. The device according to claim 1, wherein said device measures glucose levels in said human blood of said user.

7. The device according to claim 6, wherein a patient suffers from a disease caused by abnormal insulin levels in the patient's blood.

8. The device according to claim 7, wherein the patient suffers from a disease selected from the group consisting of Type I or Type II diabetes, obesity, insulin resistance, high blood pressure, peripheral neuropathy, cardiovascular disease, metabolic syndrome, kidney disease, nephropathy, stroke, Alzheimer's disease, hepatopathy, and combinations thereof.

9. The device according to claim 8, wherein the device is used to treat or manage the diseases said patient is suffering from.

10. The device according to claim 1, wherein said device comprises machine learning programming.

11. The device according to claim 1, wherein said device comprises artificial intelligence programming.

12. The device according to claim 1, comprising
one or more embedded circuit boards;
one or more sources of light;
a microcontroller or microprocessor comprising an algorithm;
an ultrasonic transducer, wherein said ultrasonic transducer is designed to measure acoustic energy;
a power source; and
a means for displaying the substance measurement value.

13. The device according to claim 12, further comprising a chassis.

14. The device according to claim 12, wherein said chassis comprises an upper clamping arm and a lower clamping arm connected by a spring biasing means;
wherein said chassis secures the device to the patient.

15. The device according to claim 14, wherein said arms further comprise grooves.

16. The device according to claim 14, wherein said clamping means is selected from the group consisting of a coil, spring, helical compression spring, extension spring, flat spring, torsion spring, clamp, hinge, pin, adhesive, gel, elastomeric material, and combinations thereof.

17. The device according to claim 12, wherein said light source is selected from the group consisting of a laser, laser diode, light-emitting diode, photodiode, an electrogenerated chemiluminescence-200 (ECL-200), multichannel light source-8000 (MLS-8000) and combinations thereof.

18. The device according to claim 17, wherein the laser diode is a Fabrey-Perot laser diode.

19. The device according to claim 18, wherein said diode is connected to a digital output pin.

20. The device according to claim 18, wherein the Fabrey-Perot laser diode transmits light as pulses.

21. The device according to claim 20, wherein said pulses are emitted at a frequency between 1 to 10 Hz±5%.

22. The device according to claim 17, further comprising a lens.

23. The device according to claim 12, wherein said power source is one or more batteries.

24. The device according to claim 23, wherein said one or more batteries are selected from the group consisting of lithium polymer batteries, rechargeable batteries, and combinations thereof.

25. The device according to claim 12, wherein said display means is an organic light-emitting diode screen.

26. The device according to claim 25, wherein said display means shows a blood glucose concentration level.

27. The device according to claim 12, wherein said microcontroller further comprises a transmitter.

28. The device according to claim 12, wherein said transducer is a piezoelectric component.

29. The device according to claim 27, wherein said transmitter is wireless.

30. The device according to claim 12, wherein said microcontroller further comprises an amplifier.

31. The device according to claim 12, wherein said microcontroller further comprises a transistor.

32. The device according to claim 12, wherein said transistor is a bipolar junction (BJT) or metal-oxide-semiconductor field-effect transistor (MOSFET).

33. The device according to claim 12, further comprising a digital output pin, wherein said transducer is connected to said pin.

34. The device according to claim 27, wherein said microcontroller takes a baseline reading of the user's tissue.

35. The device according to claim 12, wherein said circuit boards further comprise one or more embedded sensors selected from the group consisting of acceleration, inertial, humidity, temperature, barometric, pressure, proximity, light color and luminosity sensors, and combinations thereof.

36. The device according to claim 12, wherein said embedded circuit boards further comprise a microphone.

37. The device according to claim 12, further comprising a wireless interface; wherein said measured substance concentration levels can be viewed on a wireless hardware piece.

38. The device according to claim 37, wherein the wireless hardware piece is selected from the group consisting of a mobile phone, tablet, watch, wearable device, monitor, and computer.

39. The device according to claim 12, further comprising a wiring interface; wherein the device is connected by said wiring interface to a vital sign monitor capable of displaying the measured substance concentration level of the user.

40. The device according to claim 39, further comprising a means for monitoring blood oxygen concentration, wherein the measured blood oxygen concentration is displayed on the vital signs monitor.

41. The device according to claim 39, further comprising a means for monitoring body temperature, wherein the measured body temperature is displayed on the vital signs monitor.

42. The device according to claim 39, further comprising a means for monitoring pulse rate, wherein the measured pulse rate is displayed on the vital signs monitor.

43. The device according to claim 28, wherein the piezoelectric component has a frequency in the range of 3 to 5 MHz±5%.

44. The device according to claim 18, wherein the laser diode emits light energy having a wavelength in the range of 1550 to 1750 nm±5%.

45. The device according to claim 44, wherein the laser diode emits light energy having a wavelength of about 1600 nm±5%.

46. The device according to claim 12, wherein the device further comprises a resistor having a resistance of 30Ω to 35Ω±5%.

47. The device according to claim 46, wherein said resistor is installed in place.

48. The device according to claim 13, wherein the piezoelectric component, laser diode light source, microcontroller, and display screen are contained in a chassis assembly, the chassis assembly being adapted for holding a body part of a user.

49. A non-invasive blood glucose measuring device, comprising an ultrasonic transducer for measuring the baseline glucose concentration of a patient, a laser diode light source for transmitting light energy pulses into a body tissue of the patient, and a microcontroller wherein the ultrasonic transducer detects vibration of the body tissue and generates an acoustic signal that is transmitted to a microcontroller, and wherein the concentration of the blood glucose is measured using at least one algorithm that determines the difference between the baseline and final blood glucose concentration levels.

50. The blood glucose measuring device according to claim 49, wherein the intensity of the ultrasonic waves of the ultrasonic transducer is related to the intensity of the continuously changing intensity of the light applied to the body tissue.

51. A method for non-invasive measuring of blood glucose concentration levels in a patient, comprising the steps of:
measuring the baseline of the resting tissue of a user;
transmitting light energy pulses into a body tissue of the user;
detecting the vibration of body tissue resulting from the light energy pulses being transmitted, wherein the vibrations generate an acoustic signal; and
analyzing the acoustic signal using at least one algorithm to determine the difference between the baseline and final level of vibration, wherein the algorithm interprets the level of vibrations into the amount of blood glucose.

52. The method according to claim 51, further comprising the additional step of:
using a microcontroller to determine if the baseline tissue values are within a predetermined range and the algorithm proceeds if the baseline values are within this range.

53. The method according to claim 51, wherein a laser diode is used to transmit said light energy pulses.

54. The method according to claim 51, wherein the at least one algorithm determines a time interval for transmitting the light energy pulses and a time interval for pausing the light energy pulses.

55. The method according to claim 51, wherein the baseline (NL) data and light transmitted laser diode induced (L) data are saved as variables.

56. The method according to claim 55, wherein the saved variables are inserted into the algorithm equation: Signal Value=([L1+L2+L3/3)−([NL+NL2+NL3]/3).

57. The device according to claim 12, wherein said device is attached to the body tissue of a user, wherein said body tissue is relatively thin with an available blood supply.

58. The device according to claim 12, wherein said device is attached to a body part of the patient selected from the group consisting of interdigital folds, thenar webspace, one or more fingers, wrist, arm, ear, nostril, head, lip, tongue, neck, back, stomach, chest, genitals, and foot.

59. The device according to claim 14, wherein the arms of the chassis are manufactured from a polymer resin injection molding process or 3-D printing process.

60. The device according to claim 14, wherein the parts selected from the group consisting of circuit boards, piezoelectric component, and laser diode, are custom made.

61. The device according to claim 12, wherein said device incorporates embedded machine learning (ML), artificial intelligence (AI), internet of things (IoT), app, and blockchain programming, wherein said programming is designed to encrypt and/or deidentify personal data and measurements.
